(19) United States
(12) Patent Application Publication
(10) Pub. No.: US 2021/0017234 A1
(43) Pub. Date: Jan. 21, 2021

(54) DURABLE RHIZOMANIA RESISTANCE

(71) Applicant: SESVanderHave, Tienen (BE)

(72) Inventors: Mark VARRELMANN, Gottingen (DE); Veronika WETZEL, Gottingen (DE); Yann GALEIN, Tienen (BE)

(73) Assignee: SESVanderHave, Tienen (BE)

(21) Appl. No.: 16/981,193

(22) PCT Filed: Mar. 15, 2019
(86) PCT No.: PCT/EP2019/056529

§ 371 Date: Sep. 15, 2020

(30) Foreign Application Priority Data
Mar. 16, 2018 (EP) 18162178.0

(51) Int. Cl. C07K 14/005; C12N 7/00; G01N 33/00; C12N 15/82; A01N 63/40; A01N 63/50

(52) U.S. Cl. CPC C07K 14/005; C12N 7/00; G01N 33/0098; C12N 2770/00022; A01N 63/40; A01N 63/50; C12N 15/8283

(57) ABSTRACT

A method to predict and modulate the Rz2 resistance towards Beet necrotic yellowing vein virus (BNYVV) is based on the identification of variance in TGB1 from BNYVV, isolated variant TGB1 and corresponding uses, as well as the use of Rz2 sugar beets for soils infected with the beet soil-borne mosaic virus.

### FIELD OF THE INVENTION

The present invention is in the field of agronomy and of resistance towards Beet necrotic yellowing vein virus (BNYVV), responsible for the disease rhizomania, as well as the Beet soil borne mosaic virus (BSBMV) or the Beet soil-borne virus (BSBV) and discloses a method to monitor and/or predict the occurrence of Rz2 resistance-breaking BNYVV as well as a method to generate and/or select mutant Rz2 forms that confers to sugar beets a more robust/durable resistance towards rhizomania.

### BACKGROUND OF THE INVENTION

In crops, unacceptable proportions of the harvest may be lost due to virus infections.

A widespread viral disease of the sugar beet plant (Beta vulgaris) is called “rhizomania” and is caused by a Benyvirus, the Beet necrotic yellow vein virus (BNYVV), which is soil-borne and transmitted to the root of the beet by the Plasmodiophoromycete Polymyxa betae. The BNYVV genome consists of 4 or 5 RNA molecules, depending on the isolate.

The disease significantly affects acreages of the area where the sugar beet plant is grown for industrial use in Europe, Asia and USA and is still spreading, especially in Western Europe: the virus can stay dormant in a contaminated soil for more than 10 years, whereas human activities such as irrigation and displacement of infected plants or plant parts, or infected soils, result in a long-distance propagation of the disease.

One convenient way to selectively and efficiently suppress all the plant viruses is based on dsRNA constructs targeting the expression of key proteins of the virus by the mechanism of RNA silencing in transgenic plants. dsRNA constructs have been developed against BNYVV (WO2007128755) with exceptional results in field trials. These constructs are stably incorporated in the plant genome. However, such constructs, although efficient, safe and corresponding to a clear need, are not easily commercialized, which forces to find alternatives for controlling virus infections.

A first dominant major rhizomania resistance gene, called Rz1, has been identified after an extensive research effort made by the Holly Sugar Company, however the identity of the gene is still unknown. The avirulance gene, the viral protein recognized or targeted by Rz1, most probably is the protein P25, encoded by BNYVV RNA3.

However the BNYVV isolates, such as the isolates found in Pithiviers (France), carrying a fifth RNA molecule encode an additional pathogenicity factor P26 and escape the Rz1 resistance. Mutations in RNA3-encoded P25 in a specific amino acid motif (position 67-70) in certain isolates also permit the virus to overcome Rz1.

Other important rhizomania resistance sources have been identified in WB41 and WB42 lines of B. vulgaris maritima. The gene from WB42 conferring resistance towards rhizomania has been called Rz2, and the one from the line WB41 has been called Rz3.

Breeders have been able to commercialize sugar beet varieties with both Rz1 and Rz2 in tandem (Meulemans et al., 2003) so as to deliver a stronger and more durable resistance towards rhizomania. The gene Rz2 has then been identified by a consortium of research groups (Capistrano-Grossman et al., 2017: Crop wild relative populations of Beta vulgaris allow direct mapping of agronomically important genes, Nature, 2017). The Rz2 encoded proteins has the structure of a typical R-protein.

The genomic locus of Rz2 and Rz3 has been identified years ago: and it is the same, and recent research has confirmed that Rz2 and Rz3 represent the same resistance gene.

The inventors expect that there will be, sooner or later, the occurrence of a BNYVV resistant isolate towards Rz2/Rz3, as it was the case for Rz1.

Other rhizomania resistance genes have been suggested, but they are less potent and, sometimes, associated with a yield penalty.

Therefore there is a need to develop further resistance towards rhizomania on the basis of the Rz2 protein, and also to develop methods to precisely and rapidly determine if BNYVV isolates occur that are able to overcome Rz2 resistance.

Beside rhizomania, other viral infection are, or may become, problematic. Among them are the Beet soil-borne mosaic virus (BSBMV), which is related to BNYVV, and the Beet soil-borne virus (BSBV).

### SUMMARY OF THE INVENTION

A first aspect of the present invention is a method to modulate the resistance towards Beet necrotic yellowing vein virus (BNYVV) comprising to:
obtain a plant or a plant tissue expressing a functional Rz2 protein against BNYVV,
obtain a variant BNYVV TGB1 protein having between 90% and 99% of identity with SEQ. ID NO:2 and/or with SEQ. ID NO:3 and/or with SEQ. ID NO:4,
measure the interaction between this functional Rz2 protein (SEQ. ID NO:1) and this variant TGB1 protein,
obtain a plant or a plant tissue expressing a variant Rz2 protein,
measure the interaction between this variant Rz2 protein and this variant TGB1 protein and
select a variant Rz2 protein displaying an increased interaction with this variant TGB1 protein by comparison to the interaction of this functional Rz2 protein (SEQ. ID NO:1) with this variant TGB1 protein.

A related aspect of the present invention is a method to monitor the resistance of a sugar beet plant towards Beet necrotic yellowing vein virus (BNYVV), this sugar beet plant expressing a functional Rz2 protein, comprising to isolate a TGB1 (SEQ. ID NO:2) protein variant from this BNYVV and to measure the interaction between this functional Rz2 protein and this TGB1 protein variant of this BNYVV.

Preferably, in these methods, the interaction between Rz2 protein or a variant thereof and TGB1 protein or of a variant thereof is measured at the protein level, preferably by pull-down, by yeast two hybrid, by fluorescence complementation or by FRET, or in vivo, preferably by measuring a resistance response upon addition of this TGB1 protein or of this variant of TGB1 protein to a plant or a plant part expressing this functional Rz2 or this variant Rz2.

Preferably, in these methods, the functional Rz2 protein or the variant of this functional Rz2 protein shares at least 90% of identity with SEQ. ID NO:1 over the full-length of the said sequence.

Advantageously, in these methods, the variant TGB1 protein is obtained from BNYVV isolated from soils with long-term cultivation of sugar beet expressing a functional Rz2 protein.

Another related aspect of the present invention is the use of a TGB1 protein (SEQ. ID NO:2) or of a variant thereof from Beet necrotic yellowing vein virus (BNYVV) to monitor the occurrence of Rz2 resistance-breaking mutants, the said TGB1 protein variant having between 90% and 99% of identity with SEQ. ID NO:2 and/or with SEQ. ID NO:3 and/or with SEQ. ID NO:4, the said identity being measured over at least 100 consecutive amino acids.

Preferably, this variant TGB1 is isolated from soils with long-term cultivation of sugar beets expressing a functional Rz2 protein.

Another related aspect of the present invention is an isolated TGB1 obtained from Beet necrotic yellowing vein virus (BNYVV) from soils with long-term cultivation of sugar beets expressing a functional Rz2 protein.

Another related aspect of the present invention is the use of this isolated TGB1 (obtained from BNYVV from soils with long-term cultivation of sugar beets expressing a functional Rz2 protein) for monitoring the Rz2 resistance towards BNYVV.

Another related aspect of the present invention is the use of a sugar beet plant or seed expressing a functional Rz2 protein for soils infected with the beet soil-borne mosaic virus or for soils infected with the beet soil-borne virus.

In this use, preferably, the functional Rz2 protein shares at least 90% of identity with SEQ. ID NO:1 over the full-length of this sequence.

### DETAILED DESCRIPTION OF THE INVENTION

The inventors have identified the viral avirulence gene (Avr) product targeted by Rz2. The BNYVV protein is encoded by the so-called triple gene block 1, located on RNA2 and represents a movement protein with a molecular mass of 42 kDa (TGB1 or P42).

This identification was not possible by routine experiments but has been achieved through the use of a non-conventional approach.

The discovery allows to (i) monitor natural occurring BNYVV isolates for variations in this P42 Avr-protein, which variation risks to confer the ability to overcome Rz2 resistance and (ii) to identify Rz2 variants that can still be active against this resistance breaking BNYVV isolates with TGB1 variants.

The invention further allows to predict the resistance of (RZ2-expressing) sugar beet plants towards viruses having a TGB1 protein, such as BSBMV and BSBV.

Therefore, a first aspect of the present invention is a method to determine the resistance of a sugar beet plant towards Beet necrotic yellowing vein virus (BNYVV), comprising to measure the interaction between the (functional) Rz2 protein (SEQ. ID NO:1 and similar sequences) of the sugar beet and the Triple Gene Block protein 1 (TGB1) protein (SEQ. ID NO:2 and similar sequences) of the BNYVV.

Alternatively, TGB1 of the Beet soil-borne mosaic virus (BSBMV; SEQ. ID NO:3) or of the Beet soil-borne virus (BSBV; SEQ. ID NO:4) can be used, or any TGB1 protein sharing a significant (e.g. more than 80% of) identity (or of homology, for instance after a BLASTp comparison; Blosum62) with TGB1 of BNYVV and/or of BSBMV and/or of BSBV over at least 100 consecutive amino acids (of SEQ. ID NO:2 and/or SEQ. ID NO:3 and/or SEQ. ID NO:4), preferably without taking into account the 23 N-terminal amino acids of SEQ. ID NO:2 and/or the 21 N-terminal amino acids of SEQ. ID NO:3.

This (the above) sugar beet plant is expressing a functional Rz2 protein (SEQ. ID NO:1 and similar sequences).

In the context of the present invention, a “functional Rz2” protein preferably refers to a protein that is expressed, (at least in the roots, and/or at a level enough to confer resistance) sharing at least 85% of identity with SEQ. ID NO:1 over the full-length of the sequence, preferably at least 86, 87, 88, 89, 90, 91, 92, 93, 94, 95, 96, 97, 98, 99%, or even sharing 100% of identity over the full-length of SEQ. ID NO:1. The percentage of identity can be measured using BLASTp software, for instance using a BLOSUM62 matrix. A suitable functional Rz2 protein has more than 95% of identity with SEQ. ID. NO: 1 and in addition some (conserved) amino acid changes, possibly marked as positive in BLASTp (for instance using a BLOSUM62 matrix). Typical functional Rz2 proteins have 1, 2, 3, 4, or 5 mutations (substitution of amino acids, deletion and/or addition of amino acids) in SEQ. ID NO:1

Alternatively, or in addition, a suitable Rz2 protein advantageously keeps at least one, two or the three functional domains (100% of identity of these domains present in SEQ. ID NO:1), such as the CC, the NB and the LRR domains.

A preferred method to determine if a given Rz2 protein is functional (a protein sharing at least 85% of identity with SEQ. ID NO:1 over the full-length of the sequence, preferably more than 86, 87, 88, 89, 90, 91, 92, 93, 94, 95, 96, 97, 98, 99%, or even 100% of identity with SEQ. ID NO:1), is to measure at the protein level the interaction between this Rz2 and TGB1 (SEQ. ID NO:2, SEQ. ID NO:3, SEQ. ID NO:4 or any TGB1 protein sharing a significant (e.g. more than 80%) identity (or of homology, for instance after a BLASTp comparison; Blosum62) with TGB1 of BNYVV and/or of BSBMV and/or of BSBV over at least 100 consecutive amino acids). Such measurement can be achieved by pull-down, by yeast two hybrid, by fluorescence complementation or by FRET.

Another preferred method to determine if Rz2 is functional, which can be used as an alternative, or in addition to the in-silico comparison and/or the protein interaction measurement, is based on the inoculation of a construct comprising SEQ. ID NO:2 or SEQ. ID NO:3 or SEQ. ID NO:4 on a plant (sugar beet) or plant (sugar beet) part (e.g. a leaf or a tissue) comprising the Rz2 protein (but not the Rz1 protein) and on the monitoring of the resistance response (cell death and/or in situ H₂O₂ production; the resistance response can be an hypersensitive response or an extreme resistance response) developed by this plant or plant part. Only functional Rz2 will cause a resistance response when put into contact with the TGB1.

The plant carrying Rz2 can be a plant having Rz2 in its genome such as a Beta vulgaris plant, or a transformed plant (sugar beet, or a non-sugar beet plant such as Nicotiana benthamiana, or other Beta species) to express Rz2, either stably or transiently.

A preferred construct comprising SEQ. ID NO:2 (or SEQ. ID NO:3, SEQ. ID NO:4 and similar sequences) is a cDNA clone of BNYVV (or of a part thereof). Advantageously, such cDNA clone is inoculated through Agrobacterium (transient agroinfiltration).

A related aspect of the present invention is a method to modulate (improve and/or isolate improved Rz2 protein) the resistance towards Beet necrotic yellowing vein virus (BNYVV) and/or the Beet soil-borne mosaic virus (BSBMV) and/or the Beet soil-borne virus (BSBV) comprising to obtain a (sugar beet) plant or a (sugar beet) plant part expressing a functional Rz2 protein (as defined above; Preferably SEQ. ID NO:1) against BNYVV and/or against BSBMV and/or against BSBV,

to obtain variant (functional) BNYVV TGB1 proteins having between 90% and 99% of identity with SEQ. ID NO:2 and/or variant (functional) BSBMV TGB1 proteins having between 90% and 99% of identity with SEQ. ID NO:3 and/or variant (functional) BSBV TGB1 proteins having between 90% and 99% of identity with SEQ. ID NO:4, to measure the interaction between this functional Rz2 protein with a TGB1 having 100% of identity with SEQ. ID NO:2 or with SEQ. ID NO:3 or with SEQ. ID NO:4,

to obtain (and/or generate) a variant of this Rz2 (protein),

to measure the interaction between such variant Rz2 (protein) and the such variant TGB1 (protein) and to select (and isolate) the variant Rz2 (protein) displaying an increased interaction with such variant TGB1 (protein) by comparison to the interaction of the said functional Rz2 protein (SEQ. ID NO:1) with such variant TGB1 protein.

Another related aspect of the present invention is the use of TGB1 protein (SEQ. ID NO:2) and variants thereof from Beet necrotic yellowing vein virus (BNYVV) or of TGB1 protein (SEQ. ID NO:3) from Beet soil-borne mosaic virus (BSBMV) or of TGB1 protein (SEQ. ID NO:4) from Beet soil-borne virus (BSBV) to monitor the occurrence of Rz2 resistance-breaking mutants.

Preferably, the variant TGB1 of the above-methods is obtained from Beet necrotic yellowing vein virus (BNYVV) isolated from soils with long-term cultivation of sugar beets expressing a functional Rz2 protein.

Alternatively, (Rz2) variants are generated by (random or targeted) mutagenesis.

Another related aspect of the present invention is a (an isolated) TGB1 (protein, RNA or cDNA) obtained from Beet necrotic yellowing vein virus (BNYVV) isolated from soils with long-term cultivation of sugar beets expressing a functional Rz2 protein.

Preferably, this (isolated) TGB1 (protein, RNA or cDNA) obtained from Beet necrotic yellowing vein virus (BNYVV) isolated from soils with long-term cultivation of sugar beets expressing a functional Rz2 protein is a variant TGB1.

In the context of the present invention (the above methods and the TGB1 variant as such), these soils with long-term cultivation preferably refer to soils known to be infected with BNYVV, more preferably known to be infected with BNYVV isolates able to overcome Rz1-resistance.

In the context of the present invention (the above methods and the TGB1 variant as such), “long-term cultivation of sugar beets expressing a functional Rz2 protein” preferably refers to 1 year or more of culture of such Rz2-(and/or Rz3-) sugar beet, more preferably to 2, 3, 4, 5 or more years of culture.

Another related aspect of the present invention is the use of a sugar beet plant or seed expressing a functional Rz2 protein for soils containing the Beet soil-borne mosaic virus and/or for soils containing the Beet soil-borne virus.

### BRIEF DESCRIPTION OF THE FIGURES

FIG. 1: Quantification of viral content in sugar beet plants after vortex-inoculation of BNYVV and of BSBMV.

FIG. 2: Identification of the avirulence gene for Rz2 by transient expression.

### EXAMPLES

### Example 1

BSBMV Infection is Controlled by Rz2, but not by Rz1.

Generation of the Viruses

Viruses have been obtained from infected soils and propagated as well-known in the art. For instance, B. macrocarpa have been infected with either BNYVV or BSBMV. Then, the sap from systemically infected leaves was produced and used to infect young sugar beet plants, for instance by mechanical inoculation.

Infection of Sugar Beet Plants

Existing varieties of sugar beet plants with either Rz1, Rz2 (SES VanderHave, Belgium) or no resistance towards rhizomania (susc; two different varieties) have been inoculated with either BNYVV (an isolate with 4 RNAs) or BSBMV by vortex inoculation.

The abundance of the virus in the plant (infected or not) is estimated by ELISA quantification of the viral coat protein.

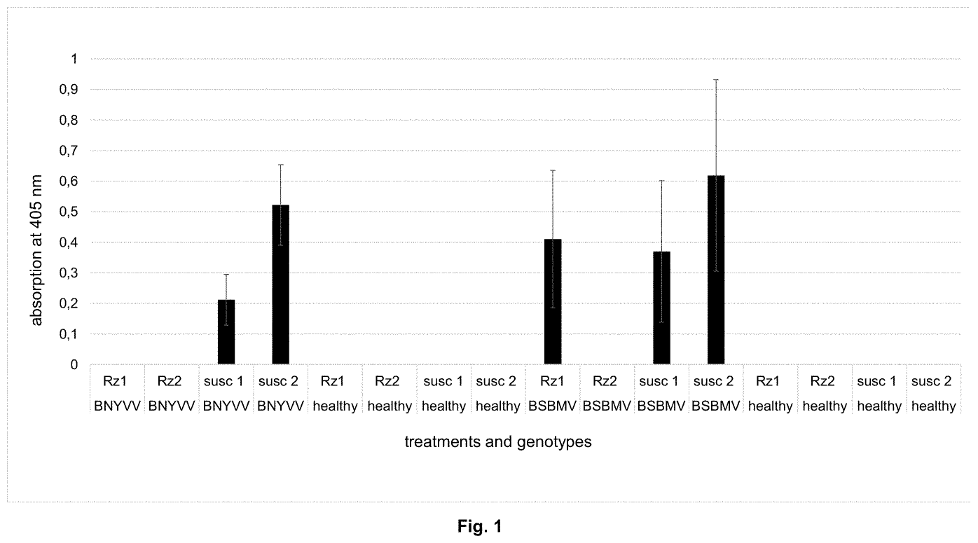

Rz1 and Rz2 efficiently control the infection with BNYVV (FIG. 1). Rz2, in addition, provides a strong protection against infection with BSBMV or BSBV.

Moreover, the clones that have been developed are shown to correctly reflect the underlying physiology, and can thus be used for further experiments, such as infection experiments of Rz2-containing plants.

### Example 2

Identification of the Avirulence Protein of BNYVV and of BSBMV

The inventors have expressed several constructs of BNYVV and/or of BSBMV in Beta plants expressing a functional Rz2 protein and no functional Rz1 and checked in plantar, after 3,3′-diaminobenzidine (DAB) staining, for an immune response.

DAB Staining

Rationale: H₂O₂ is produced during resistance response, such as a hypersensitive (immune) reaction. The polymerization product of DAB in contact with H₂O₂ results in a strong-reddish brown color, which is visible to the naked eye.

Protocol: 0,1% DAB-PBS buffer is infiltrated by vacuum into detached leaf-samples. Incubation for at least 3 hrs; boiling of samples in 96% ethanol to bleach samples.

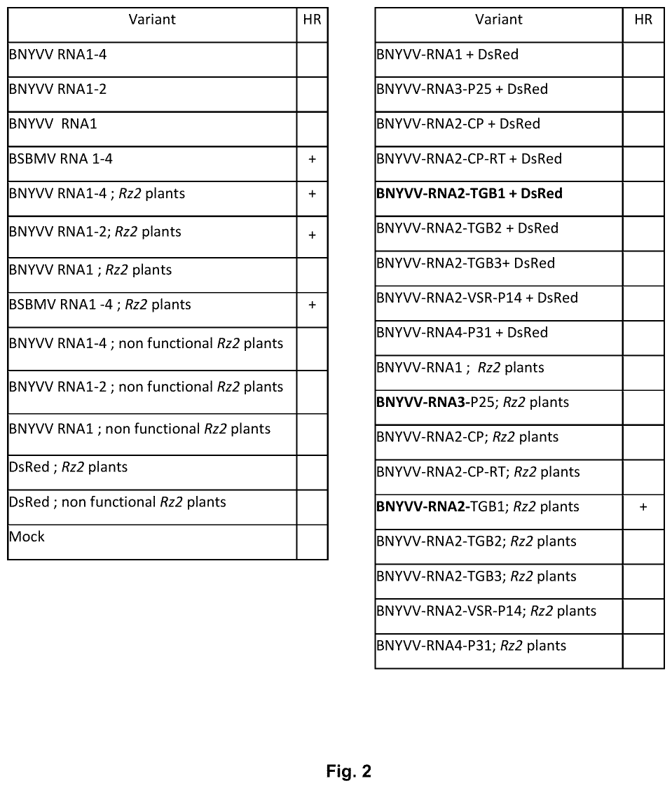

The expression of RNAs 1-4 of BNYVV and of BSBMV causes a major resistance response in Rz2 (not Rz1) plants and no resistance response in non-Rz2 plants (FIG. 2, left column).

A similar resistance response was observed in Rz2 plants infiltrated with only RNA 1-2 of BNYVV.

However, the expression of RNA 1 alone did not elicit immune resistance response in Rz2 plants.

Then the inventors identified that only TGB1 (SEQ. ID.NO:2) of BNYVV (and not the other proteins expressed by this virus) caused immune resistance response in Rz2 plants (FIG. 2, right column). This protein is necessary and sufficient for this phenomenon in these plants.

1. A method to modulate the resistance towards Beet necrotic yellowing vein virus (BNYVV) comprising:
obtaining a plant or a plant tissue expressing a functional Rz2 protein against BNYVV,
obtaining a variant BNYVV TGB1 protein having between 90% and 99% of identity with SEQ. ID NO:2 and/or with SEQ. ID NO:3 and/or with SEQ. ID NO:4,
measuring interaction between the said functional Rz2 protein (SEQ. ID NO:1) and the said variant TGB1 protein,
obtaining a plant or a plant tissue expressing a variant Rz2 protein,
measuring interaction between the said variant Rz2 protein and the said variant TGB1 protein and
selecting a variant Rz2 protein displaying an increased interaction with said variant TGB1 protein by comparison to the interaction of said functional Rz2 protein (SEQ. ID NO:1) with said variant TGB1 protein.

2. A method to monitor resistance of a sugar beet plant towards Beet necrotic yellowing vein virus (BNYVV), said sugar beet plant expressing a functional Rz2 protein, comprising isolating a TGB1 (SEQ. ID NO:2) protein variant from said BNYVV and measuring interaction between said functional Rz2 protein and said TGB1 protein variant of said BNYVV.

3. The method of claim 1, wherein the interaction between Rz2 protein or a variant thereof and TGB1 protein or of a variant thereof is measured at the protein level, by pull-down, by yeast two hybrid, by fluorescence complementation or by FRET, or in vivo, by measuring a resistance response upon addition of said TGB1 protein or of said variant of TGB1 protein to a plant or a plant part expressing said functional Rz2 or said variant Rz2.

4. The method according to claim 1, wherein the functional Rz2 protein or the variant of said functional Rz2 protein shares at least 90% of identity with SEQ. ID NO:1 over the full-length of said sequence.

5. The method according to claim 1, wherein the variant TGB1 protein is obtained from BNYVV isolated from soils with long-term cultivation of sugar beet expressing a functional Rz2 protein.

6. A method of using a TGB1 protein (SEQ. ID NO:2) or of a variant thereof from Beet necrotic yellowing vein virus (BNYVV) comprising monitoring occurrence of Rz2 resistance-breaking mutants, said TGB1 protein variant having between 90% and 99% of identity with SEQ. ID NO:2 and/or with SEQ. ID NO:3 and/or with SEQ. ID NO:4, said identity being measured over at least 100 consecutive amino acids.

7. The method of claim 6, wherein the variant TGB1 is isolated from soils with long-term cultivation of sugar beets expressing a functional Rz2 protein.

8. An isolated TGB1 obtained from Beet necrotic yellowing vein virus (BNYVV) from soils with long-term cultivation of sugar beets expressing a functional Rz2 protein.

9. A method of using the isolated TGB1 of claim 8 comprising monitoring the Rz2 resistance towards BNYVV.

10. A method of using a sugar beet plant or seed expressing a functional Rz2 protein for soils infected with a beet soil-borne mosaic virus or for soils infected with the beet soil-borne virus.

11. The method of claim 10, wherein the functional Rz2 protein shares at least 90% of identity with SEQ. ID NO:1 over the full-length of said sequence.

12. A method of using a sugar beet plant or seed expressing a functional Rz2 protein (SEQ. ID NO:1) for soils infected with a beet soil-borne mosaic virus or for soils infected with the beet soil-borne virus.


SEQUENCE LISTING (4 sequences)

SEQ ID NO: 1 - protein, 1163 aa, Beta vulgaris
  DOMAIN (168)..(227): NBS
  DOMAIN (591)..(613): LRR
  DOMAIN (1013)..(1072): CC
MDVVGTALSAAQSLFAALQSSELKEILSIFGYKSRLDDLQRTVSTINAVFRDAETKQELTHEAQHWLEELKDAVFEADDLFDEFVTLAEQKQLVEAGGSLSKKMRQFFSDSNPLGIAYRMSRGVKKIKKKLDAIAYNHQFSFKIDLEPMKERRLETGSVVNAGDIIGREDDLEKIVGLLLDSNIQRDVSFLTIVGMGGLGKTALAQLVYNDPRVRTAFPLRCWNCVSDQDQKQLDVKEILGKILATATGKNHEGSTMDQVQTQLREQLCGKRYLLVLDDVWNENPNQLRDLVEFFMGGRSRNWIVVTTRSHETARIIRDGPLHKLQGLSEENSWRLFVRWTFGSVQAKFPNDFIMIARDIVDKCARNPLAIRVVGSLLCGQDKSKWLSFHEIDLGNIRKSHNDIMPILNLSYHHLEPPIKRCFSYCAVFPKDFLIGKQTLINLWMAQGYIVPLDKDQSIDDASEEYISILLRRCFFENVGAEKDGVIKIHDLMHDIAQNVMGKELCTTKNISGSLDKSVRHLSLARTSFARYSFNATHIRSYFCAGYWCQDAEINQFSVEALVPNCLYLRAMDLAWSKIKSLPDSIGGLLHLRYLDLSYNEDLEVLPNSIAKLYNLQTLQLKGCKRLEGLPKHLSRLVKLQTLDIHGCNNVTYMPKGMGKLTCLHTLSKFIVGGEGSCSSWKQCFDGLEDLKALNNLKGHLEIQIRWPKNTTDAVKEDVTREGLYLNHKEHLNHIVVDFRCEEGGGRMDDEEARRLMEELRPHPYLENLAVKAYYGVKMPGWATLLPNLTELFLSDCGELENLPCLGNLDHLKVLRLSHLAKLEYIEEDSSSANFRCRPGPESAGLSLYFPSLERLELKRLCKLKGWRRGEGLGDDHQPFNESSSNTQVQLQLCLPQLKSLRIERCPLLTFMPLCPKTEKLHLVVFNERLRIVHAKRDENFYAPLHSSSSDPENPRNTIPIPMFREVYINNVAWLNSLPMEAFRCLTHMTIKNDEVESLGEVGEVFRSCSSSLRSLNITGCSNLRSVSGGLEHLTALEMLEIYDTHKLSLSEDPEGVVPWKSLHHSLSYLQLMNLPQLVNLPDSMQFLAALRTLSIVHCTKLQSVPDWMPRLTSLRKLMVSFCSAHLERRCQNPTGVDWPNIQHIPSIDVTSSLPKFLVLPYE

SEQ ID NO: 2 - protein, 384 aa, Beet necrotic yellow vein mosaic virus
  MISC_FEATURE: TGB1
MVQVQRRTGGDKGAKGNYASSAPVRSRRMTQDDWSRTHPDDIFSVIEKTLVEDGYKWNGVKPGHCDWGKLKESGAIDNFRGTLEGELGKNCDLTCNAAAVKLDTLQKVKMSSDWTARVGIVLGAPGVGKSTSIKNLLDKFGAKHKMVLCLPFSQLLEGVFAGRLDTFLVDDLFCRSVEYGKYNTMLVDEVTRVHMCEILVLAGHLGVKNVICFGDPAQGLNYKAGSAVNYNFPIIAECYASRRFGKATADLINSSNGGGKPVVGNNEVKDSWTFEELCGKILDMSTVLVATRETQKFLLEDNIESILYSDAHGQTYDVVTIILEDEFDDAAICDPNVRAVLLTRARKGGMIKMGPNIAARFKNGDFNSRGVSKSCTGDTFCEDR

SEQ ID NO: 3 - protein, 382 aa, Beet soil-borne mosaic virus
  MISC_FEATURE (1)..(382): TGB1
MAPEQHKQNASETASGRRNSSVRSRGMSKDDWSVTHPDDVFSIIEKTLVEDGYKWHGVKPGHCDWDKLEQSGAIKNFKGTLEGEVDSSCSLTCNAAAIKLDIVERLDVSSDWSARVGIVLGAPGVGKSTSIKHILDTYGSRYKMVLCLPVKQLLDGVFSGRMDTFLIDDIFSRSVDYGKYHTMLVDEITRVHMCEVLVLAGYLGIKNVICFGDPAQGINFKAGSAVNYNFPVIAECYSSRRFGVATADLINSCNGGGKSVVGNNDVKDNWTFEELCGKIEEMSTVLVATHATKEFLADDGIEAVYYEDAQGMTYDVVTIVLKDEFDDDAICDSNVRAVLLTRARKGGLLKVDPNIAARFKNGDFNSRGVSKACTGDTFCEDR

SEQ ID NO: 4 - protein, 427 aa, Beet soil-borne virus
  MISC_FEATURE (1)..(427): TGB1
MEKEKLQKKEKNLNRRTNKKGVRRSDKKNSERKTKEDNRKLVEEDVADDLVAHESVAKDRAATCERGESQRQRADQETSVKEGAGVDSKLGSDRYAGKRQLEVVSRICQESGFVATGKPLKRYPEDYFLKSGLLADFDKYLSDRLDKGCNLTKSETETVLKHLRQKRRPQSFLAGSVTGVPGSGKTTLLRKIQTEAGLNSVVILANERHKIRFTQLPACYTAKEILLLRTAIKYDVLLIDEYTLLQNGEILLLQRILEAKVVVLFGDRAQGNSRTADSPEWLQIPVIYSSVKSRRFGKATADFCGKQGFDFEGCDQEDEVQKLDFEGSSPETDINLALTEATIEDLKEVGIECSLVKDVQGNEYDSVSLFIREEDRAALSDPELRSVAFTRHRKLLIVRIPVCLMLSLFNGELNSDYRPQTNHYGKN